(19) United States
(12) Patent Application Publication
(10) Pub. No.: US 2008/0292677 A1
(43) Pub. Date: Nov. 27, 2008

(54) Engineered lung tissue, hydrogel/somatic lung progenitor cell constructs to support tissue growth, and method for making and using same

(75) Inventors: Joaquin Cortiella, Galve Ston, TX (US); Larry Bonassar, Ithaca, NY (US); Koji Kojima, Brookline, MA (US); Joan E. Nichols, Galveston, TX (US); Ronald P. Mlcak, Bayou Vista, TX (US); Jean A. Niles, Galveston, TX (US)

Correspondence Address: ROBERT W STROZIER, P.L.L.C, PO BOX 429, BELLAIRE, TX, 77402-0429, US

(73) Assignee: THE BOARD OF REGENTS OF THE UNIVERSITY OF TEXAS SYSTEM

(21) Appl. No.: 11/298,543

(22) Filed: Dec. 9, 2005

Related U.S. Application Data
(60) Provisional application No. 60/634,563, filed on Dec. 9, 2004.

(51) Int. Cl. A61K 35/42; A61K 9/10; C12N 5/06; A61F 2/00; A61F 2/02

(52) U.S. Cl. 424/423; 424/93.7; 435/396; 435/378

(57) ABSTRACT

Somatic lung progenitor cell/polymer constructs are disclosed along with methods for isolating somatic lung progenitor cells from adult mammals, seeding the cells onto or into polymeric scaffolds and allowing the cells to differentiate and proliferate into functional lung tissue/polymer implants. A method for treating lung disease, disorders or injuries is also disclosed.

### BACKGROUND OF THE INVENTION

1. Field of the Invention

The present invention relates to novel functional pulmonary tissue constructs adapted to restore pulmonary functions to non-functioning portion of diseased or damaged mammalian lungs including human lungs and to method for making and using same.

More particularly, the present invention relates to novel functional pulmonary tissue constructs or compositions adapted to restore pulmonary functions to non-functioning portion of diseased or damaged mammalian lungs including human lungs and to method for making and using same, where the compositions include a mixture of lung derived somatic progenitor cells grown on a bio-compatible, bio-degradable polymeric scaffold.

2. Description of the Related Art

Diseases of the lung, including chronic obstructive pulmonary diseases (COPD) such as emphysema, chronic bronchiolitis, and asthmatic bronchitis, are collectively the fourth leading cause of death in the world, with an annual healthcare cost of $24 billion in the United States alone. Current treatment of COPD or other respiratory disorders frequently relies on antibiotics, bronchodilators, and oxygen therapy to minimize discomfort and optimize function in the remaining lung tissue. While lung transplantation remains the only viable option for many terminally ill COPD patients, the success of this approach is limited by 1) long-term complications of immunosuppression resulting from the transplant procedure; and 2) the overall shortage of available donor tissue (which results in many patients on the transplant list dying before an appropriate tissue-matched organ is found).

A potential solution to the problem caused by organ shortages is the production of new functional replacement tissues using tissue-engineering techniques. Tissue engineering has shown great promise for the generation of a variety of tissues (including bone, cartilage, liver and pancreas) for which organ donation shortages currently exist. Tissue engineering of the lung, however, has not progressed as rapidly, with only a few published reports focusing on the growth of the airway epithelial cells on synthetic polymer substrates. While these studies have generated initial enthusiasm about the potential for lung therapies, the actual engineering of all of the component parts of lung tissue has been limited. The slow progress in this area may be due to the complexity of the tissue and the variety of cell types present in functional lung, including epithelial cells, smooth muscle cells, endothelial cells, and specialized pneumocytes. There are two approaches that utilize progenitor cell populations to promote the growth of functional complex tissue that could be employed to provide for this cellular diversity: 1) the use of multipotent somatic precursor cells capable of differentiating into progeny with multiple differentiation phenotypes, and/or 2) the use of mixtures of unipotent somatic progenitor cells, each giving rise to an array of lung specific single-cell lineages.

Despite constant environmental assaults and attacks by respiratory pathogens, the lung maintains an ability to restore and regulate homeostasis and reestablish a normal state after injury or insult. The capacity of pulmonary progenitor cells to repair and regenerate tissue suggests that there is a balance between stem cell renewal and differentiation, even in a relatively nonregenerative organ such as the lung. Recent reports have documented the identification of novel stem or progenitor cells exhibiting extraordinary plasticity from a variety of adult mammalian tissues (including fat, deciduous teeth, skin, muscle, and bone marrow) that have given rise to multiple cell lineages. Relatively little is known about stem and progenitor cells that exist in the lung or the process of their differentiation and organization into lung tissue. However, several recent works have described potential sources of progenitor cells capable of generating some of the cellular components of lung tissue. In one study, mesenchymal stem cells injected intravenously into lethally irradiated mice were shown to engraft into alveoli and bronchi and express lung-specific markers. Another study documented the ability of lung and bone marrow-derived cell populations with the SP phenotype to develop into lung alveolar epithelial cells. Embryogenic stem cells have also been shown to give rise to type II alveolar epithelial cells, but few references support the possibility that organ-derived lung progenitor cells have the capacity to develop into lung tissue. A mammalian, organ-specific, spore-like cell has also been shown to have the capacity to develop into pancreas or liver tissue (depending on the source of the cell), and there is a suggestion that a spore-like cell isolated from the lung may have the potential to generate lung tissue. Historically, several epithelial cell types (including Clara cells, pulmonary neuroendocrine cells, basal cells, and type II pneumocytes), all with the unipotent potential to give rise to an array of lung-specific single cell lineages, have been identified in adult lung.

Specifically, it has been suggested that pulmonary neuroendocrine cells or neuroendocrine bodies contribute to airway repair after injury and may also serve as a reservoir of progenitor cells capable of epithelial regeneration. But multipotent pulmonary stem or progenitor cells capable of differentiating into progeny with multiple differentiation phenotypes, including those cells with unipotent transient amplification potential, have not yet been identified for the lung.

Thus, there is a need in the art for a composition and methodology for multipotent pulmonary stem or progenitor cells capable of differentiating into progeny with multiple differentiation phenotypes.

### DEFINITIONS OF THE INVENTION

The term SLPCs somatic lung progenitor cells and the term SPLCs mean somatic progenitor lung cells. These terms have exactly the same meaning and are used interchangeably throughout the text.

The term SLPC/polymer construct means a three-dimensional (3D) polymer network into which SLPCs have been seeded.

The term scaffold means a three-dimensional (3D) polymer network into which SLPCs can be seeded.

### SUMMARY OF THE INVENTION

The present invention provides novel functional pulmonary tissue constructs or compositions adapted to restore pulmonary functions to non-functioning sites of diseased, damaged or injured mammalian lungs including human lungs, where the compositions include a mixture of lung derived somatic progenitor cells grown on a bio-compatible, bio-degradable polymeric scaffold.

The present invention also provides a composition including a bio-compatible, bio-degradable polymeric scaffold supporting a pulmonary functional tissue derived from a mixture of lung derived somatic progenitor cells.

The present invention also provides an implantable composition including a bio-compatible, bio-degradable polymeric scaffold supporting a pulmonary differentiated and functional tissue derived from a mixture of lung derived somatic progenitor cells

The present invention provides a method including the step of isolating a composition including a mixture of lung derived somatic progenitor cells obtained from an autologous biopsy. The isolated lung progenitor cells are then deposited or seeded onto and/or into a polymer scaffold preferably a hydrogel scaffold to a form a cell/polymer construct adapted to grow new and functional pulmonary tissue. The cell/polymer construct is then allowed to develop into a functional pulmonary tissue/polymer construct. The tissue/polymer construct is then implanted into non-functioning areas of a diseased lung to restore some or all of the functionality of the non-functioning areas. Alternatively, the cell/hydrogel construct can be directly implanted into non-functioning areas of a diseased lung, where cell differentiation and proliferation into functional pulmonary tissue occurs in vivo to restore some or all of the functionality of the non-functioning area. This treatment utilizes adhesion and gel properties of hydrogels such as Pluronic F-127 available from Sigma-Aldrich Corp., St. Louis, Mo., to aide stem cell differentiation and tissue growth in non-functioning areas of a mammalian lung including a human lung. The cell/hydrogel constructs allow for the creation of new engineered and fully functional pulmonary tissues that will enhance oxygenation and reduce occurrence of ventilatory problems associated with emphysema, COPD and other lung disorders.

The present invention also provides a method including the step of isolating a composition including a mixture of lung derived somatic progenitor cells obtained from an autologous biopsy. The isolated lung progenitor cells are then deposited or seeded onto and/or into a polymer scaffold preferably a hydrogel scaffold to a form cell/polymer construct adapted to grow new and functional pulmonary tissue. The cell/polymer construct is then allowed to develop into a functional pulmonary tissue/polymer construct. The tissue/polymer construct is then implanted into non-functioning areas of an injured lung to restore some or all of the functionality of the non-functioning area. Alternatively, the cell/hydrogel construct can be directly implanted into non-functioning areas of a diseased lung, where cell differentiation and proliferation into functional pulmonary tissue occurs in vivo to restore some or all of the functionality of the non-functioning area. The construct can also be implanted into damaged or injured lung tissue sites to promote healing, ameliorate adverse symptoms, prevent further lung damage and to protect the sites during healing.

### DESCRIPTION OF THE DRAWINGS

The invention can be better understood with reference to the following detailed description together with the appended illustrative drawings in which like elements are numbered the same:

FIG. 1 depicts a schematic diagram of methods for isolation, culture, implantation and growth of tissue from somatic lung progenitor cells (SLPCs);

FIGS. 2a-i depict phenotypic characterization of ovine-derived somatic lung progenitor cells with CD45, Lineage-1, and major histocompatibility complex class I and class II staining are shown as solid histograms, immunoglobulin isotype matched control antibody for each stain are shown as red line overlay and control levels were equal to or less than 1% positive for all controls using data from 10,000 cells collected for each sample;

FIGS. 3A-F depict phase contrast micrographs of freshly isolated SLPCs (A) and the cells after culture for 4 days (B), 1 week (C) and 3 weeks (D), while (E) depicts western blot analysis for Clara cell protein 10 (CC10) and (F) depicts western blot analysis for surfactant protein C (SP-C) in an extract of normal lung tissue and progenitor cell cultures on days 0, 4, 7, and 14;

FIGS. 4A-C depict immunocytochemical analyses of in vitro differentiated cultures showing CC10, SP-C and cytokeratin production by ovine lung derived somatic progenitor cells on Days 0 and 14 of culture showing expression of these mature lung markers on Day 14 of culture;

FIGS. 5A-G depict scanning electron micrographs of in vitro tissue-engineered lung ovine somatic lung progenitor cells seeded onto polyglycolic acid scaffolds and an in vitro engineered lung tissue macroscopically visible by 8 weeks of growth;

FIGS. 6A-C depict sections of engineered lung, produced after implantation of ovine somatic lung progenitor cell (SLPC)/polymer tissue constructs on the backs of nude mice;

FIGS. 7A-F depict sections of engineered lung, produced after implantation of ovine SLPC/PF-127 tissue constructs on the backs of nude mice;

FIGS. 8A-D depict immunohistochemical detection of Clara cell-10 in normal tissue and in (implanted ovine somatic lung progenitor cell/polymer tissue constructs with arrows indicating positive staining and the specificity of immunohistochemical analyses was demonstrated by confirming that no evidence of reactivity was obtained in the absence of the primary antibody;

FIGS. 9A-D depict evaluation of Masson trichrome staining of tissue engineered ovine lung in nude mouse implanted ovine stem cell/polymer tissue constructs showing hematoxylin and eosin staining of normal lung and tissue engineered lung and Masson trichrome staining of normal lung and tissue engineered lung;

FIGS. 10A-G depict in vivo tissue engineered ovine lung using polyglycolic acid (PGA) or pluronic F-127 (PF-127) cell/polymer constructs using autologous ovine carboxyfluorescein diacetate, succinimidyl ester (CMFDA) labeled somatic lung progenitor cells (SLPCs) prior to implantation showing the CMFDA fluorescent labeling of cells with strands of PGA in the background and sections of engineered lung, produced after implantation of ovine SLPC/polymer tissue constructs at the lung wedge resection site; and

FIGS. 11A-D depict an in vivo tissue engineered ovine lung using somatic lung progenitor cell/polyglycolic acid (SLPC/PGA) construct showing implantation of construct after a pneumonectomy at the right main stem bronchus site.

### DETAILED DESCRIPTION OF THE INVENTION

The inventors have found that synthetic polymers progenitor cell/polymer constructs such as polyglycolic acid (PGA) and Pluronic F-127 (PF-127) progenitor cell/polymer constructs can be produced to support lung tissue development in both in vitro and in vivo human model system. The inventors have demonstrated that these precursor cells can differentiate into numerous cell types that produce Clara cell protein 10 (CC10), cytokeratin, and surfactant protein C (SP-C) prior to formation of cell/polymer constructs. The inventors have also shown that the use of synthetic polymers such as PGA and PF127 not only facilitated tissue formation, but aids in proper tissue assembly.

Although the in vitro studies using PGA to engineer lung tissue were promising, PGA was not the polymer of choice for developing lung tissue in vivo as it created a foreign body response that effected tissue growth. The inventors have isolated and characterized a population of adult-derived or somatic lung progenitor cells from adult mammalian lung tissue and demonstrated the promotion of alveolar tissue growth by these cells (both in vitro and in vivo) after seeding onto synthetic polymer scaffolds. After extended in vitro culture, differentiating cells expressed both Clara cell 10 kDa protein, surfactant protein-C, and cytokeratin, but did not form organized structures. When cells were combined with synthetic scaffolds, polyglycolic acid (PGA) or Pluronic F-127 (PF-127), and maintained in vitro or implanted in vivo, they expressed lung-specific markers for Clara cells, pneumocytes, and respiratory epithelium and organized into identifiable pulmonary structures (including those similar to alveoli and terminal bronchi) with evidence of smooth muscle development.

While PGA has been shown to be an excellent polymer for culture of specific cell types in vitro, using PGA in in vivo culture constructs, in immunocompetent hosts, induces a foreign body response that altered the integrity of the developing lung tissue or alters the integrity of the implanted engineered lung tissue. On the other hand, pulmonary cell constructs using PF-127 as the polymer scaffold in the constructs resulted in the development of tissue with a smaller inflammatory response when implanted into immunocompetent hosts. Thus, the preferred construct for enhancing the therapeutic use of engineered tissues, without foreign body adverse responses, requires both the use of specific cell phenotypes as well as a synthetic polymers which either do not induce a foreign body response to induces only a minimal foreign body response in order to facilitate the assembly of functional tissue at the sites of implantation.

The present invention relates to isolated and characterized adult (somatic)-lung progenitor cells and the generation of functional pulmonary tissues such as alveolar tissue, where the tissues are derived from the cells after the cells are seeded onto a synthetic polymer scaffold to form a cell/polymer implant.

The present invention relates to a tissue engineered treatment that utilizes a composition including a mixture of lung derived somatic progenitor cells obtained from an autologous biopsy. The isolated lung progenitor cells are used to form cell/polymer constructs, and preferably cell/hydrogel constructs, where the construct are capable of producing engineer new and functional tissue prior to or after implantation into non-functioning sites of a diseased or injured lung. This treatment utilizes adhesion and gel properties of hydrogels such as Pluronic F-127 available from Sigma-Aldrich Corp., St. Louis, Mo., to aide stem cell differentiation and tissue growth in non-functioning areas of a mammalian lung including a human lung. The cell/hydrogel constructs allow for the creation of new engineered and fully functional pulmonary tissues that will enhance oxygenation and reduce occurrence of ventilatory problems associated with emphysema, COPD and other lung disorders.

Current treatments for COPD or other respiratory disorders frequently rely on antibiotics, bronchodilators, and/or oxygen therapy to minimize discomfort and optimize function in the remaining lung tissue. Treatments such as lung volume reduction surgery to restore normal function to regions of the lung damaged by COPD have been reported, but have not been shown to offer survival benefit over regular medical therapies for the majority of patients. While lung transplantation remains the only viable option for many terminal COPD patients, the success of this approach is limited by long-term complications of immunosuppression and lack of available donor tissue. Unfortunately due to the overall shortage in organ donations, many patients on the transplant list die before an appropriate tissue matched organ is found.

A potential solution to the problem caused by organ shortages is the production of new functional replacement tissues using tissue-engineering techniques. The process of tissue engineering involves the isolation and growth of a patient's autologous cells on a biodegradable and nontoxic carrier matrix to produce a polymer/cell construct followed by the delivery of the construct or of engineered tissues back into the patient. By maintaining the cells in a three-dimensional orientation during growth and development, appropriately configured engineered tissue constructs can be formed. Tissue engineering has shown great promise for the generation of a variety of tissues for which organ donation shortages currently exists, including bone, cartilage, liver, and pancreas. However, there has been little investigation of the engineering of lung tissue, with only a few reports focusing on the growth of airway epithelial cells on synthetic polymer substrates.

As described more fully herein, cells initially isolated from adult sheep lung were very small with size ranging from about 3 μm to about 611 m containing very little cytoplasm and did not express known markers of differentiated lung cells such as Clara Cell protein-1, surfactant protein A or C or neuroenolase (NEUN). Initial characterization of this cell population showed that they were. CD34+, CD117+(C-KIT), CD 135+ and in cells isolated from a murine system also stem cell antigen-I+(sca-1). After extended in vitro culture, cells expressed both Clara cell 10 kDa protein (CC10), surfactant protein-C (SP-C), and cytokeratin but did not form organized structures. When combined with synthetic hydrogel scaffold (Pluronic F-127 from Sigma-Aldrich Corp., St. Louis, Mo.) and maintained in vitro or in vivo they expressed lung specific markers for Clara cells, pneumocytes, and respiratory epithelium and organized into identifiable pulmonary structures including alveoli and terminal bronchi with evidence of smooth muscle.

Using such isolated cells, a tissue engineered treatment utilizing the cells seeded onto or into a polymer scaffold, preferably a hydrogel scaffold for treating diseased or injured lung tissue sites. The constructs are ideally suited for enhancing oxygenation and reduce the occurrence of ventilatory problems associated with emphysema, COPD and other lung disorders and for treating lungs damaged by chemical agents, heat, or other lung damaging agents.

Non-limiting examples of suitable biocompatible, biodegradable polymers, include hydrogels, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), chitin, chitosan, and copolymers, terpolymers, or higher poly-monomer polymers thereof or combinations or mixtures thereof. The preferred biodegradable polymers are all degraded by hydrolysis.

### PREFERRED METHODS OF THE INVENTION

All animal studies were performed under the guidelines of the Institutional Animal Care and Use Committee of the University of Massachusetts Medical School or the University of Texas Medical Branch at Galveston. Study animals were handled within the guidelines established by the American Physiological Society and the National Institutes of Health.

### Isolation of Mammalian Ovine SLPCs

Lung tissue was acquired in the form of discarded tissues, which were obtained from protocols that did not use chemical or biological agents. Lungs from adult female sheep (average weight=32 kg) or 2- to 4-month-old male Balb/C mice were harvested and washed in Dulbeco's phosphate-buffered saline solution (DPBS) with antibiotics (streptomycin [90 mg/mL], penicillin [50 mU/mL] and the antimycotic amphotericin B [25 μg/mL]; Gibco Industries, Inc., Langley, Okla.) and were frozen at −70° C. for later use.

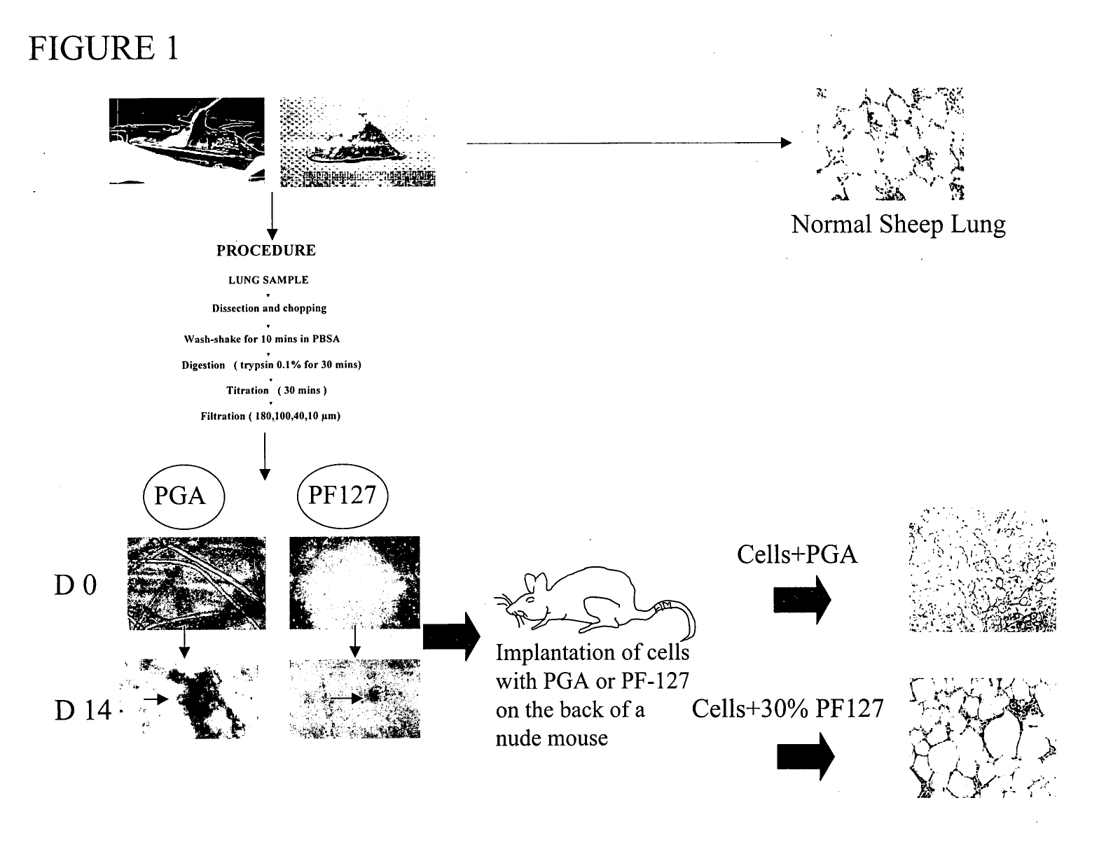

Frozen tissue was later thawed in a 37° C. water bath and washed with phosphate-buffered saline solution (PBS), and the pleura was removed. After washing, the tissue was cut into 1-cm pieces, and 0.25% trypsin was added. Tissue samples were kept in the trypsin solution for 30 min on a rocking platform and were then minced into small pieces prior to tritration using a series of progressively smaller pipette bores (FIG. 1). The remaining lung sample was passed sequentially through 100 μm, 40 μm, and then 10 μm cell strainers and washed three times in PBS with antibiotics/antimycotic (as described above). The final cell pellet was resuspended in 20 mL of complete media comprising DMEM/F12 (Gibco Industries, Inc., Langley, Okla.), which includes 10% heat inactivated fetal calf serum (FCS) (Hyclone), streptomycin (90 mg/mL), penicillin (50 mU/mL), amphotericin B (25 μg/mL), 33 mM glucose (Sigma-Aldrich Corp., St. Louis, Mo.), 20 mg/mL insulin (Sigma-Aldrich Corp., St. Louis, Mo.), 10 mg/mL transferin (Sigma-Aldrich Corp., St. Louis, Mo.), 100 nM selenium (Sigma-Aldrich Corp., St. Louis, Mo.), 10 mM putrescine (Sigma-Aldrich Corp., St. Louis, Mo.) and growth factors (20 ng/mL epidermal growth factor [EGF]) (PeproTech, Inc., Rocky Hill, N.J.) and 20 ng/mL FGF (Collaborative Biomedical, Bedford, Mass.).

In a subset of experiments, isolated ovine SLPCs were labeled with carboxyfluorescein diacetate, succinimidyl ester (CMFDA) as follows. Cells were labeled by culturing isolated adult lung cells with CMFDA solution (Molecular Probes, Eugene, Oreg.) at a concentration of 2.5 μM in RPMI 1640 media (Sigma-Aldrich Corp., St. Louis, Mo.) for 8 min at 37° C. (1×10⁷ cells/mL).

After incubation, cells were washed with RPMI-1640 at 4° C. and placed in a 175-mL flask at a concentration of approximately 5×10⁷ cells/mL, either alone or seeded onto a synthetic polymer scaffold composed of a non-woven mesh of PGA (Albany International Research Co., Mansfield, Mass.)¹⁰ or PF-127 (Sigma-Aldrich Corp., St. Louis, Mo.) and then incubated at 37° C. with 5% CO₂ for up to 8 weeks.

### Characterization of Ovine SLPCS

Live SLPCs were stained with antibodies for ovine leukocytes (cluster of differentiation antigen [CD]45, clone CO.46D5), monocyte-macrophages (CD14, clone VPM65) and T-lymphocytes (CD2, clone 1/35a) (Research Diagnostics Inc., Flanders, N.J.) as well as for Lin-1 (a mixture of antibodies directly conjugated to FITC, PE or PerCP which are specific for mature hematopoietic cells containing anti-CD14 [macrophages], -CD3 [T cells], -CD19 [B cells], -CD20 [B cells], and -CD56+16 [natural killer cells]; Pharmingen). Characterization of major histocompatibility complex (MHC) expression of isolated SLPCs was done using antibodies specific for MHC class I (clone VPM19) and class II (clone VPM36) (Research Diagnostics Inc.). Aliquots of 5×10⁵ cells were incubated with the primary antibodies described (1:250 dilutions of each primary antibody above) for 1 hour at 4° C., washed three times with PBS, and incubated for 1 hour with a secondary antibody such as fluorescein-conjugated rabbit antimouse or mouse antirabbit IgG 1:200.

For evaluation of CD34 and CD117, aliquots of 5×10⁵ cells were incubated with anti-CD34 antibody (clone 581) conjugated directly to phycoerytherin and then with anti-CD117 (clone YB5.B8) conjugated to peridinin chlorophyll protein (PerCP) as described by the manufacturer (BD Biosciences Pharmingen, San Diego, Calif.). Flow cytometry acquisition, cell sorting and analysis was done using a FACSort (Becton Dickinson, Mountainview, Calif.) using Cellquest software (Becton Dickinson). Calibration of the equipment for the validation of logarithmic linearity was accomplished using Spherotech Rainbow particles (Spehrotech). For isolation of cell populations based on size, forward versus side light scatter was used for gating. Sorting after staining, based on cell phenotype was done by anchor gating on the fluorescent population to be collected or on unstained cells.

### Western Blot

Normal sheep lung tissue and cell cultures were lysed and extracted in 50 mM Tris-HCl pH 8.0, 150 mM NaCl, 1% TritonX-100 and protease inhibitors. Extracts were loaded to 15 μg/lane, separated via SDS/PAGE on a 10% gel, blotted on to an Immobilon-P membrane blocked with 3% bovine serum albumin (BSA). To determine whether or not these cells expressed lung-specific markers, cell culture extracts from representative flasks were probed via Western blot using antibodies for CC10 (courtesy of Dr. Gurmukh Singh, University of Pittsburgh) and SP-C (Santa Cruz Biologicals). Blots were incubated at 40° C. overnight with the primary antibody (CC10 rabbit polyclonal at 1:1000 dilution or SP-C goat polyclonal at 1:100 dilution), washed, incubated for 1 hour at room temperature with an HRP-conjugated secondary antibody (1:20,000 dilution of goat anti-rabbit for antiCC10, and 1:10,000 dilution of donkey anti-goat for anti-SP-C), and visualized by chemiluminescence.

### Electron Microscopic Evaluation of In Vitro Differentiated Slpcs

Preparation of Specimens for Morphologic Examination Using Scanning Electron Microscopy was done as follows: Each tissue specimen was placed in Karnovsky's fixative for 4 hours at 4° C. The specimens were then washed with 0.1M sodium cacodylate buffer (pH 7.4), post-fixed for 1 hour in 1% osmium tetroxide, and dehydrated in increasing concentrations of ethanol. The specimen was dried in a critical point dryer with supercritical CO². The specimens were then sputter-coated with gold and observed in a Hitachi scanning electron microscope to examine the surface of the cell/polymer constructs. Portions of lung from the original donor served as normal controls.

### Evaluation of In Vitro Differentiated SLPCS

Evaluation of CC10, SP-C, and cytokeratin was done on aliquots of SLPCs taken from in vitro cultures on days 0 and 14. Cells were fixed in 2% paraformaldehyde in DPBS and permeabilized using 0.1% tritonX-100 in Hank's balanced salt solution for 2 min with a CC10 (1:40 dilution of goat anti-human CC10 IgG) (Santa Cruz Biologicals), SP-C (1:40 dilution of goat anti-human SP-C IgG) (Chemicon) or a pan cytokeratin antibody (a 1:100 dilution of mouse anti-cytokeratin AE1/AE3 IgG) (Vector Laboratories, Burlingame, Calif.) for 2 h at room temperature, and exposed to a 1:1500 dilution of Texas Red-conjugated secondary IgG (Vector Laboratories) for 30 min. Counterstaining with the blue fluorescent nucleic acid stain 4,6-diamidino-2-phenylindole, dihydrochloride (DAPI) (Molecular Probes) was done for 15 min using a 3-μM solution of DAPI in staining buffer (100 mM Tris, pH 7.4, 150 mM NaCl, 1 mM CaCl₂, 0.5 Mm MgCl₂, 0.1% Nonadet P-40).

### Implantation of Ovine Slpcs in Nude Mice

In order to support the proper development of the newly generated lung tissue, experiments were performed under conditions that would provide for the vascularization of the engineered tissues. CMFDA-labeled ovine SLPC/polymer constructs (PGA or PF-127) were created and inserted on the back of a nude mouse. Ovine SLPC/polymer constructs were created by seeding 5×10⁷ cells/mL, CMFDA labeled SLPCs in a 30% solution of the reverse thermosetting polymer hydrogel PF-127 or a 3 cm square of PGA mesh. SLPC/polymer constructs were then cultured for 24 h at 37° C. and 5% CO₂. After overnight incubation, 30 athymic mice (Charles River) then received halothane anesthesia and a subsequent 250 μL subcutaneous injection of the SLPCpolymer construct; In all experiments the following controls were used: a) PGA or PF-127 not seeded with cells b) cells suspended in saline. Samples were harvested 3 weeks after implantation and were analyzed using special stains and for lung-specific markers by immunohistochemistrying standard light microscopy and confocal microscopy.

Implantation of Ovine Progenitor Cells into a Wedge Resection Site

Adult female sheep (32 kg) were anesthetized with intramuscular ketamine (5 mg/kg) and halothane anesthesia. An open lung biopsy was performed after surgical preparation and incision. A lung biopsy sample (8 cm) was removed and placed in PBS with antibiotics. SLPCs were isolated as described above. After isolation, the cells were cultured for a period of 2 weeks in DMEM/F12 (Gibco Industries, Inc., Langley, Okla.) containing streptomycin (90 mg/mL), penicillin (50 mU/mL), amphotericin B (25 μg/mL), 33 mM glucose (Sigma-Aldrich Corp., St. Louis, Mo.), 20 mg/mL insulin (Sigma-Aldrich), 10 mg/mL transferin (Sigma-Aldrich), 100 nM selenium (Sigma-Aldrich), 10 mM putrescine (Sigma-Aldrich) and growth factors (20 ng/mL EGF) (PeproTech, Inc., Rocky Hill, N.J.) and 20 ng/mL FGF (Collaborative Biomedical, Bedford, Mass.) at 37° C. and 5% CO₂. After 2 weeks, cell/polymer constructs were created by seeding autologous lung CMFDA labeled cells at a concentration of 5×10⁷ cells/mL into a 30% solution of PF-127 or into a 30% solution of PF-127 poured onto a 3 cm square of PGA mesh.

At this time, the animal was again given ketamine and halothane anesthesia and a wedge resection was performed, removing a 10-cm portion of lung tissue from a right middle lobe; the cell/hydrogel composite was then placed into the resection site and the area was surgically closed. The animal was allowed to recover for a period of 3 weeks and was then sacrificed. Samples of lung tissue from the area where the cells were initially placed were removed for histological analysis performed using both frozen and paraffin preparations.

Implantation of PGA/SLPC Constructs after Pneumonectomy

An adult female sheep was anesthetized as described above and the right lung was removed up to the main stem bronchus. SLPCs were isolated and the cells were cultured for 2 weeks in the presence of EGF and fibroblast growth factor (FGF) while the sheep recovered from surgery. 24 hours prior to implantation, the SLPC/PGA constructs were formed by culturing the SLPC population on a 5 by 8 inch piece of PGA mesh. The P SLPC/PGA construct was sewn to the bronchial stub and the animal was allowed to recover. The animal was maintained for 3 months, after which it was sacrificed and the developing tissue was removed for gross pathologic and histologic examination.

Evaluation of Tissue Sections Derived from In Vivo Implanted SLPC/PF-127 Constructs

Evaluation of CC10, SP-C, and cytokeratin expression in normal and engineered tissue was performed as follows. Paraffin-embedded sections (5 μm) were deparaffinized in xylene, rehydrated in decreasing concentrations of ethanol, washed in tap water for 5 minutes, and incubated in 2% H₂O₂ in methanol for 30 minutes. Sections were incubated with a rabbit anti-human CC10 (1:250 dilution), a goat anti-human SP-C (1:40 dilution) or a mouse anti-pan cytokeratin (clone AE1/AE31:100 dilution) for 2 hours, washed with PBS-BSA, and then exposed to a 1:200 dilution of biotin-conjugated secondary IgG in blocking buffer for 30 min. Finally, sections were washed with PBS-BSA, incubated with avidin-biotin-peroxidase reagent for 30 minutes, washed again with PBS-BSA, exposed to diaminobenzidine for 2 minutes, rinsed in tap water for 5 minutes, and counterstained with hematoxylin for 3 minutes.

### Experimental Results Section

Isolation of the SLPC Population from Adult Mammalian Lung

Lungs from adult female sheep (average weight, 32 kg) were harvested, frozen at −70° C. and the lung tissue was enzymatically disassociated and a mixture of SLPCs was size fractionated by passing it through a series of filters as shown in FIG. 1. After isolation, SLPCs were maintained in vitro alone or seeded onto a synthetic polymer scaffold composed of a non-woven mesh of PGA or Pluronic-F127 for up to 8 weeks.

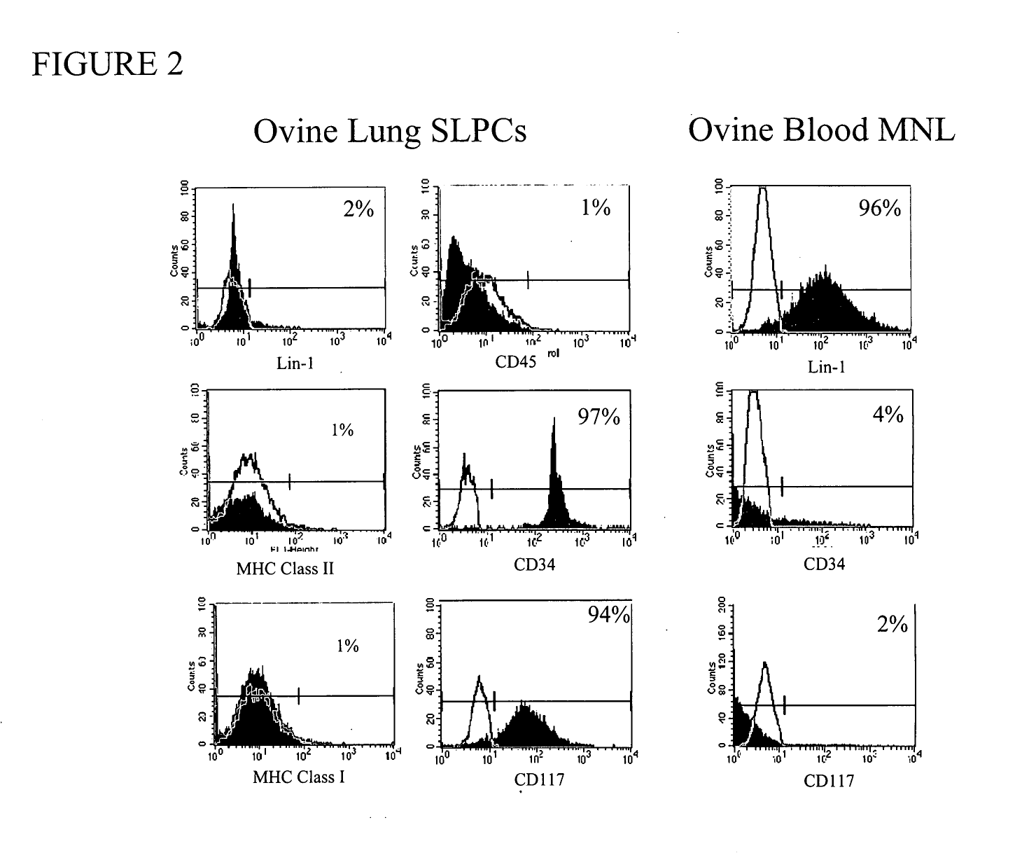

Immunophenotypic staining of freshly isolated SLPCs from adult ovine lung showed negative staining to a very low level staining for CD45 and Lin-1 as well as MHC class II as shown in FIGS. 2A-I. There was also low level expression or little expression of MHC class I, suggesting that the SLPCs were a developmentally immature cell population again as shown in FIG. 2. Using anti-human CD34 and CD117 antibodies, which cross react with ovine CD34 and CD117, the ovine SLPCs were shown to be highly positive (>90% positive), while, as expected, peripheral blood leukocytes had very low level staining for both of these stem cell markers also shown in FIG. 2.

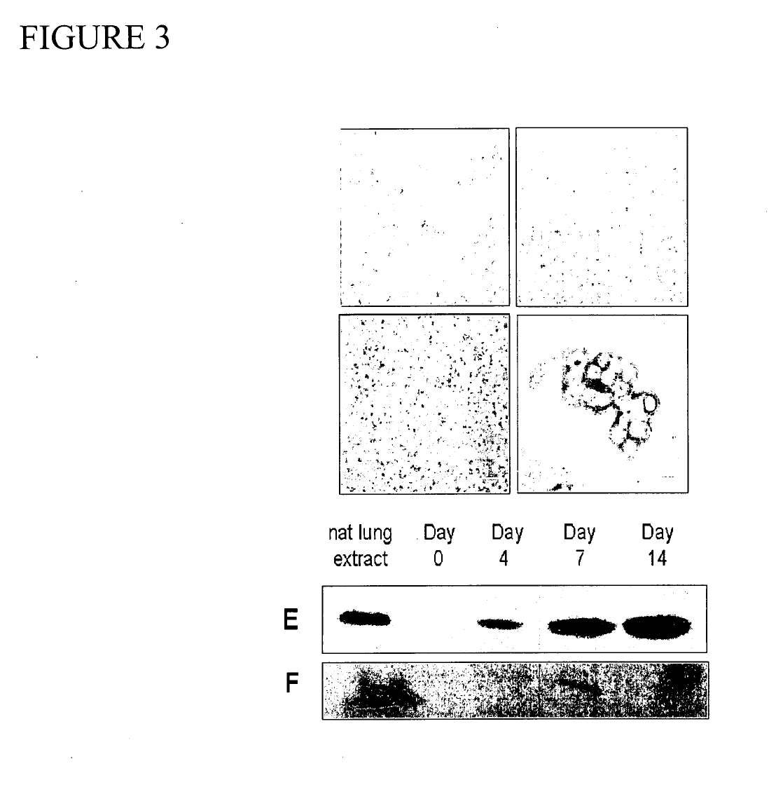

Flasks containing cells alone existed primarily as a suspension and proliferated well, with few cells attaching to the tissue culture flasks. After 4 days, less cellular debris was seen, resulting in a culture primarily composed of very small cells. After 1 week, some large cells were apparent, although the population comprised primarily of cells ranging is size from about 4 μm to about 6 μm as shown in FIG. 3A. After 3 weeks in culture, large cells formed aggregates reminiscent of neurospheres derived from neural stem cell culture as sown in FIGS. 3C and 3D. Evaluation of SP-C as shown in FIG. 3E and CC10 as shown in FIG. 3F by Western blot analysis showed that, immediately after isolation, the selected cell population did not express these markers of differentiated lung, but showed increasing levels of these markers with time in culture.

Isolated SLPCs were able to be cultured without inducing differentiation of cells for 15 passages. SLPCs remained undifferentiated and quiescent until treated with heat inactivated FCS, growth factors, EGF and FGF. CMFDA-treated SLPCs were cultured with and without addition of EGF and FGF and samples were analyzed at days 1, 3, 7 and 21 of culture for loss of fluorescence (which is indicative of cell division). Without addition of growth factors, 13% of the cells were shown to have gone through at least one round of cell division by day 21 as compared to 33% for growth factor-treated cells and 75% for the transformed cell line, HEP-2 (data not shown).

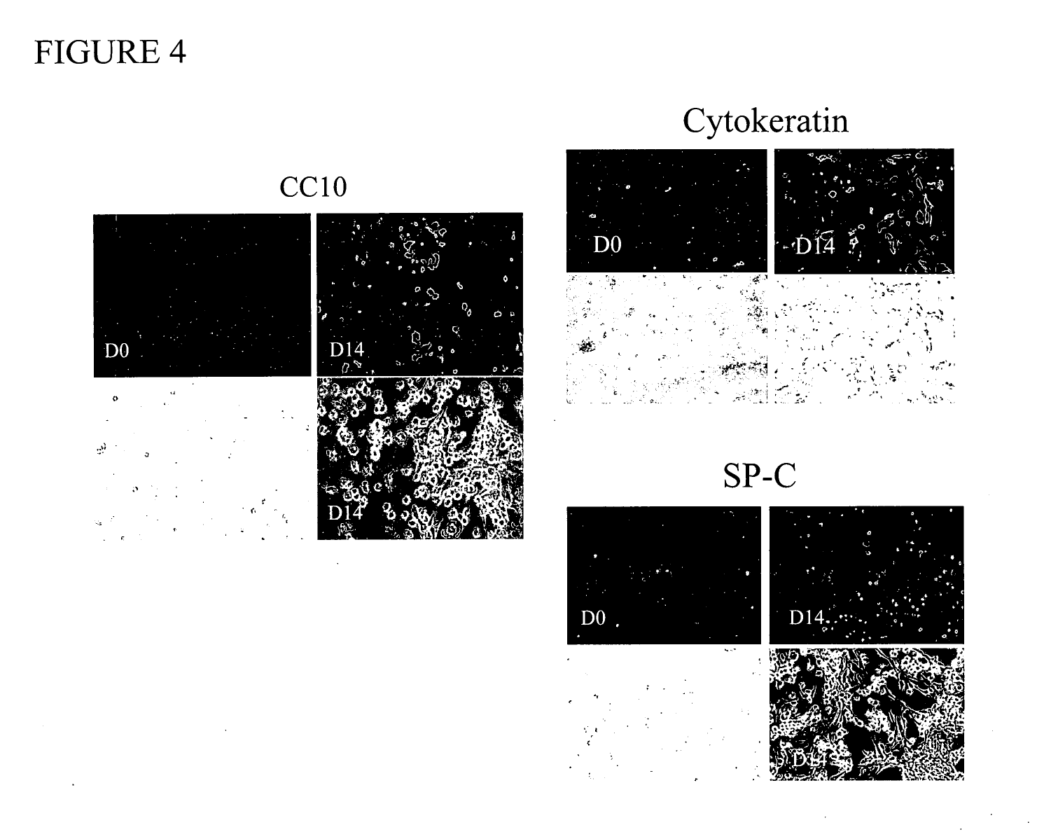

To determine whether SLPCs are capable of generating multiple cell types, isolated SLPCs were cultured in the presence of growth factors EGF and FGF, for 14 days. The 10 kd CC10 protein was previously used as a lung marker to study the growth and development of Clara cells in fetal lung. Evaluation of CC10, cytokeratin and SP-C by immunohistochemistry showed that, immediately after isolation, the selected cell population did not express these markers of differentiated lung as shown FIG. 4. After 14 days of differentiation in culture, a subpopulation of cells expressed SP-C, a marker of mature type II pneumocytes as well as CC10 and cytokeratin.

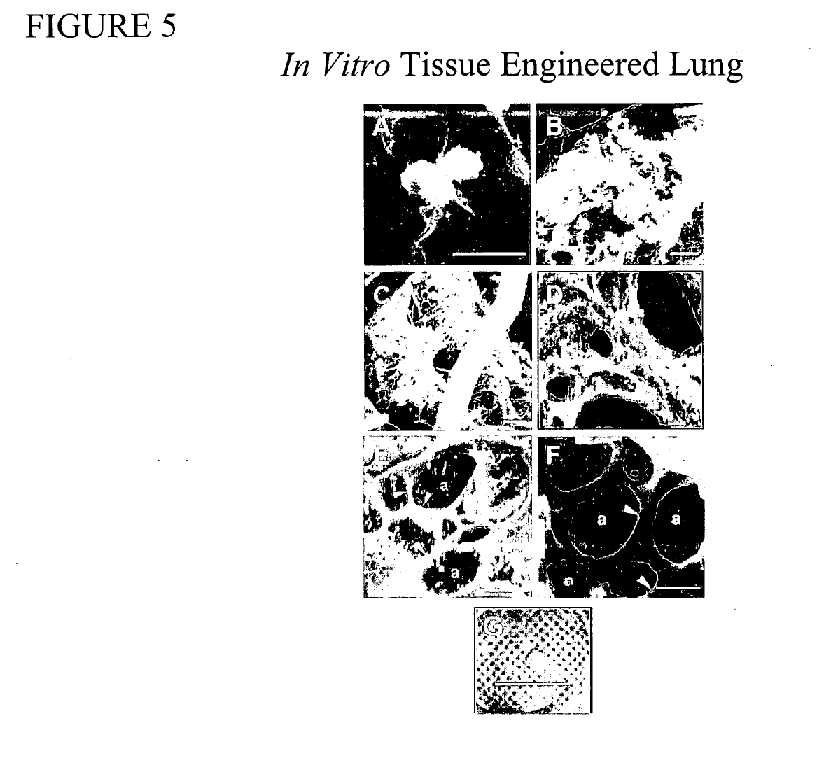

Referring now to FIG. 5, the isolated ovine SLPCs growing on PGA matrix is shown. The SLPCs maintained in flasks containing synthetic PGA scaffolds began to attach to the PGA fibers on day 1, as revealed by scanning electron microscopy (SEM). After 1 week in culture, attached cells had spread and enlarged slightly, with a small amount of extracellular matrix (ECM) synthesis evident as shown in FIG. 5B. After 2 weeks, cells had become encased in abundant ECM as shown in FIG. 5C, and, at the same time, the biodegradable PGA matrix showed signs of hydrolysis. The cells and remaining polymer were obscured by ECM after 6 weeks, and showed apparent organization into structures reminiscent of alveoli as shown in FIG. 5D. By 6 weeks, samples had developed distinct alveolar morphology as shown in FIG. 5F. The gross architecture of the engineered tissue as revealed by SEM was spongy and similar to that of normal sheep lung as shown in FIGS. 5F and 5F. Sufficient ECM production and tissue development was present to make the construct macroscopically visible after 8 weeks of culture on PGA as shown in FIG. 5G. This 4-mm piece of engineered tissue was isolated and the developing tissue was analyzed using SEM as shown in FIG. 5E.

### In Vivo Progenitor Cell Growth and Differentiation

Two-dimensional cultures of developing tissue is limited due to lack of oxygen and nutrient circulation because of the lack of appropriate vascularization. Due to these limitations, and in order to generate the necessary vascular support of the engineered tissue, ovine SLPC/polymer constructs were implanted on the backs of nude mice. Nude mice were used for these experiments due to their ability to support vascularization of developing tissue and their inability to mount an immune response against the xenographic transplant and reject the engineered tissue construct. In later experiments, autologous ovine lung cells were isolated and autologous SLPC/polymer constructs were reimplanted in an adult sheep at the site of a wedge resection.

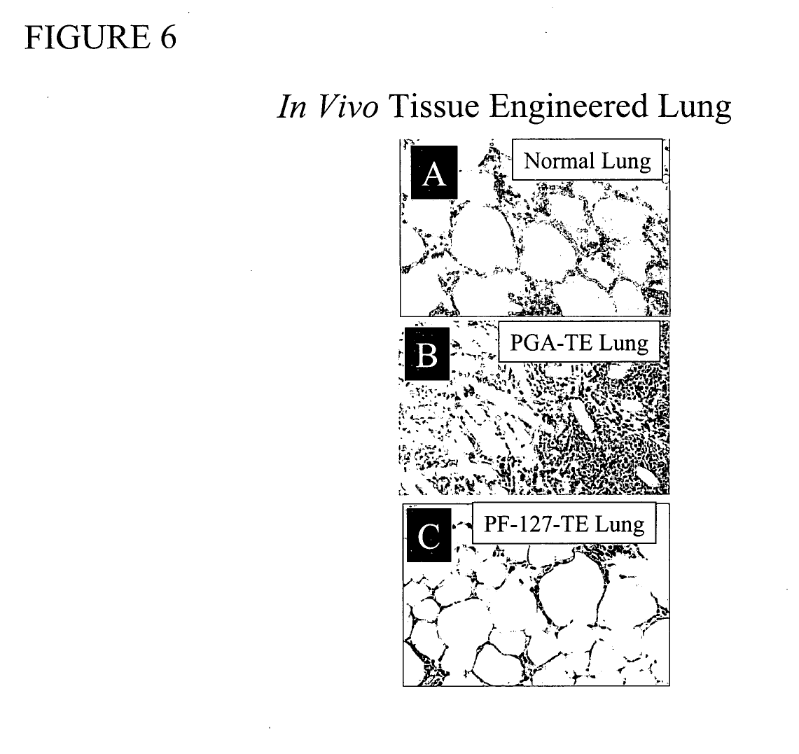

Implantation of SLPCs, PGA, or PF-127 alone generated no detectable tissue at the time points selected for tissue harvest. Implanted ovine SLPC/PGA or SLPC/PF-127 cell-polymer constructs were allowed to grow on the backs of nude mice for 3 weeks. A comparison between hematoxylin and eosin stained normal ovine lung and engineered lung isolated from the explants is shown in FIGS. 6A-C. There are gross similarities in the morphologic structure of the engineered as shown in FIG. 6C and normal lung tissues as shown in FIG. 6A at this stage of development. Looking at FIG. 6B, however, a foreign body response was induced by the PGA as is indicated by the influx of immune cells into the areas surrounding the fibers.

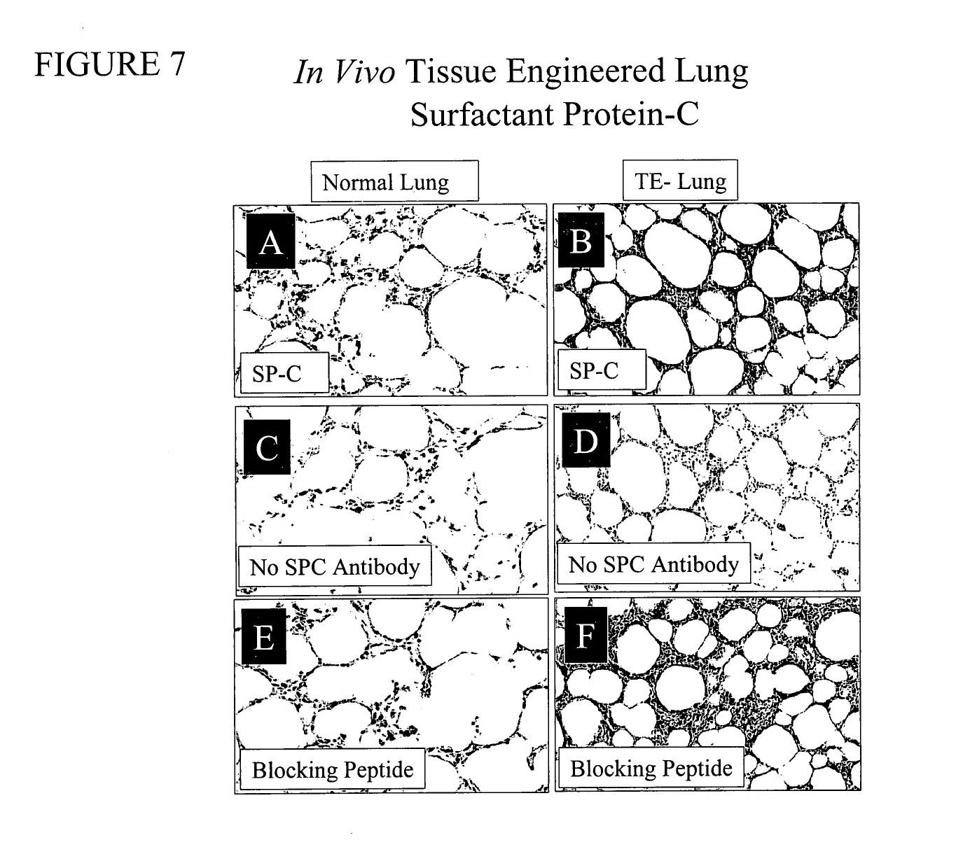

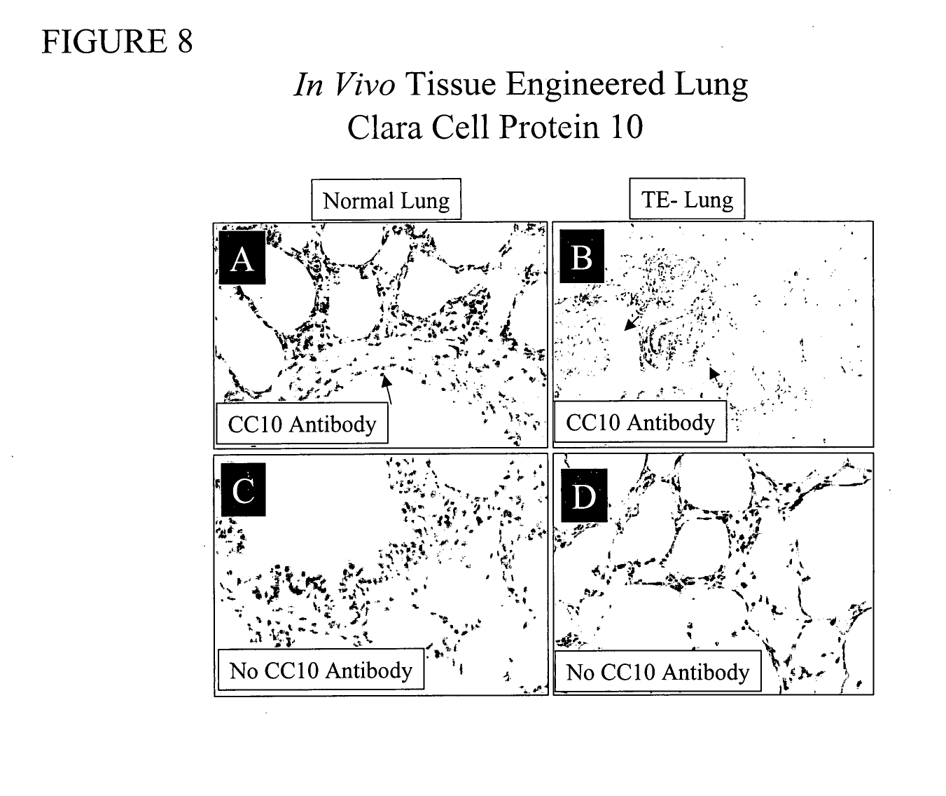

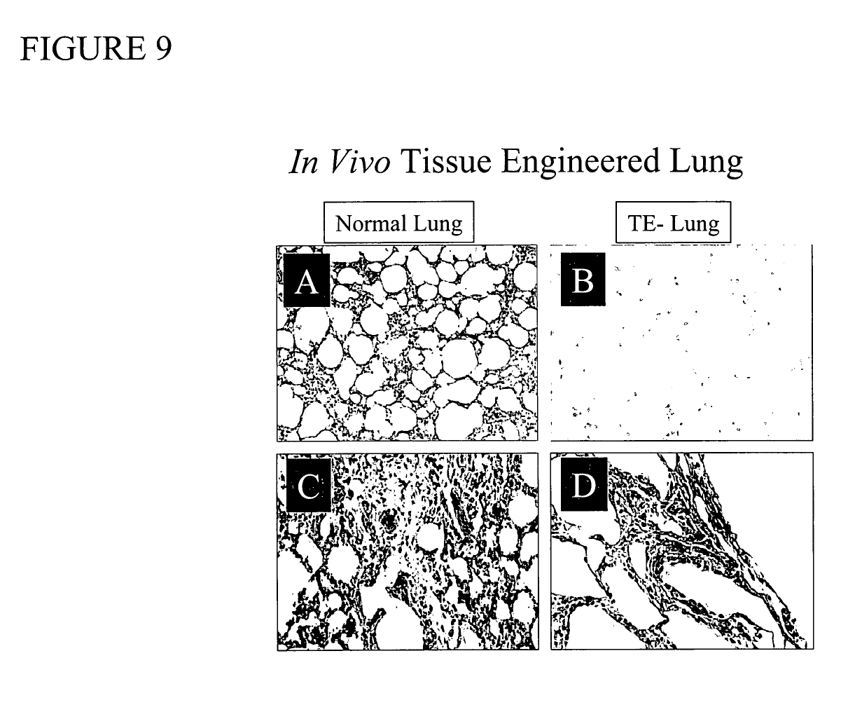

Type II pneumocytes were identified using an anti-SP-C antibody. There were higher levels of staining in tissue-engineered lung as shown in FIG. 7D compared with normal lung as shown in FIG. 7A. Cells throughout the alveolar region of the engineered tissue were positive for SP-C in both normal and engineered lung. Specificity of immunohistochemical analyses was demonstrated by confirming that no evidence of reactivity was obtained in the absence of the primary antibody as shown in FIGS. 7B and 7E or for SP-C staining in the presence of a 30-fold excess of an appropriate blocking peptide as shown in FIGS. 7c and 7f. Immunostaining of tissue sections stained with an anti-CC10 antibody showed reactivity in areas of terminal bronchi in both tissue-engineered as shown in FIG. 8b and normal lung as shown in FIG. 8a as compared to controls as shown in FIGS. 8c and 8d. Terminal bronchi in tissue-engineered lung were also less abundant and smaller than in normal lung. Detection of cytokeratin using a pan-cytokeratin antibody revealed the presence of respiratory epithelium lining incipient terminal bronchi in engineered tissue as well as more mature bronchi in normal lung (data not shown). Masson trichrome staining indicated that tissue-engineered lung specimens as shown in FIGS. 9B and 9D contained organized patterns of collagen and smooth muscle similar to native lung as shown in FIGS. 9A and 9B. Interestingly, although the tissue-engineered lung contained blood vessels, the degree of vascularization was less than in normal lung.

### In Vivo Implantation of Ovine SLPCS

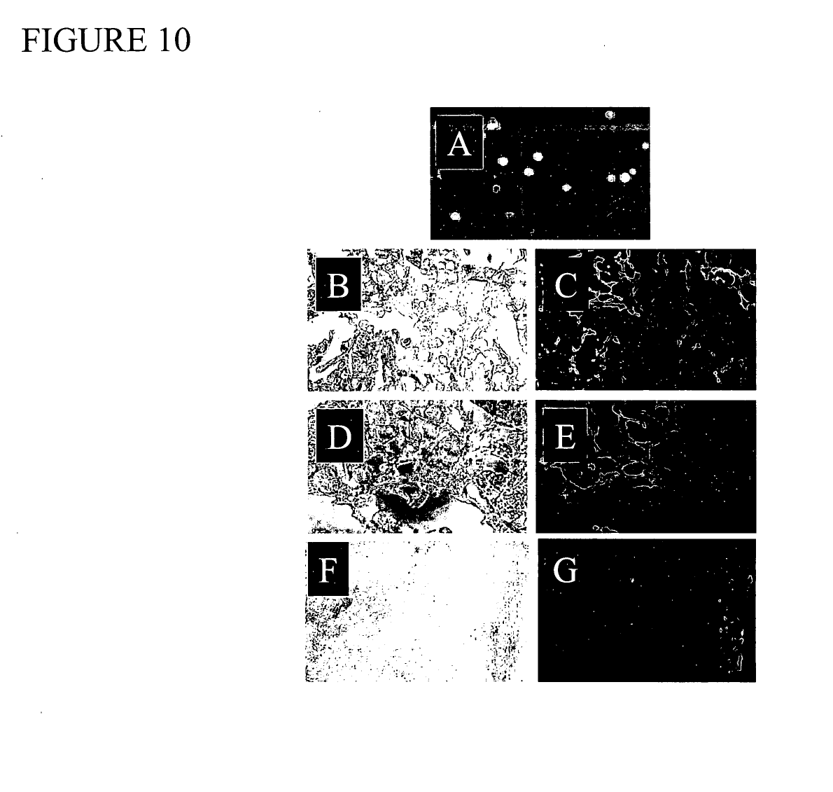

SLPCs stained with CMFDA showed a mixture of cells of sizes in the range of about 4 μm to about 6 μm as shown in FIG. 10A. The CMFDA-labeled SLPCs were combined with either the PF-127 and polymer PGA as shown in FIGS. 10B and 10C or PF-127 alone as shown in FIGS. 10D and 10E and implanted in the right upper lobe of the lung, engrafted in the area of implantation, and developed into what was easily identified upon gross examination as lung tissue as shown in FIGS. 10B-E. Phase contrast microscopy of sectioned tissue showed areas containing PGA fibers among the newly generated tissue as shown in FIG. 10B. In order to show that the normal lung parenchyma did not stain with residual CMFDA, the borders of the normal tissue and the newly engineered lung tissue were removed for histological evaluation. The edges of the normal lung tissue were joined to the engineered tissue segment in the area where the original wedge resection was done, and only the area easily identified as being within the borders of the resection was positive for CMFDA staining as shown in FIG. 10G.

### In Vivo Implantation of SLPC/PGA Construct

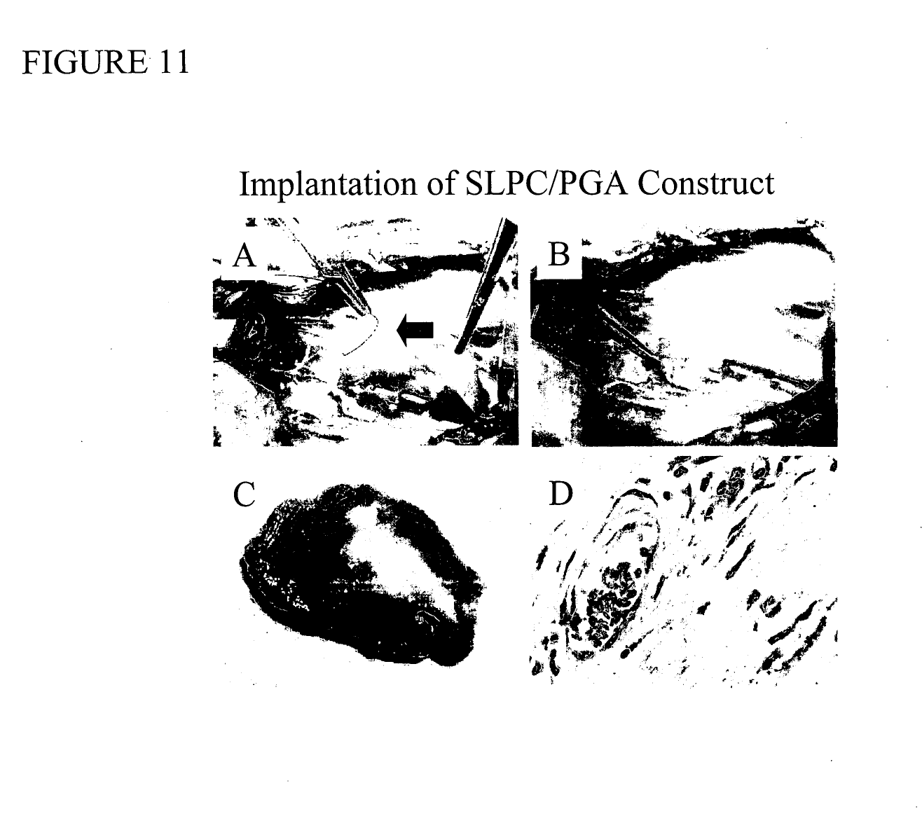

After a pneumonectomy, the PGA mesh containing the SLPCs (SLPC/PGA construct) was placed into the thoracic cavity and attached to the main stem bronchus as shown in FIGS. 11A and 11B. After 3 months the animal was sacrificed and a (5×13) fleshy soft fragment of tissue was removed form the original insertion site as shown in FIG. 11C. Sections of the tissue generated showed a fibrotic outer capsule, areas of connective tissue containing fibroblasts, and collagen fibers with limited angiogenesis but no development of normal lung architecture as shown in FIG. 11D.

### Experimental Discussion Section

The data presented here support four major conclusions. First, the data demonstrate that a somatic stem cell population can be isolated from mammalian lung. Second, these SLPCs can differentiate into numerous cell types, including, but not limited to, smooth muscle and mature cells producing CC10, SP-C, cytokeratin. Third, the data illustrates that tissue assembly is facilitated in vitro by the use of the synthetic polymers PGA and PF-127. Finally, the data illustrates that, despite promising in vitro studies using PGA to engineer lung tissue, PGA is not the polymer of choice for the development implantable of lung tissue constructs in vivo. The ability to support tissue development by PGA in vivo is very likely constrained by the development of a foreign body response to the matrix, resulting in an inflammatory reaction to the matrix material that detrimentally alters the lung tissue morphogenesis.

Initial ovine cell isolation yielded SLPCs with minimal MHC class I expression and no Lin-1 (mature monocytes-macrophages, T or B cells), CD45, or MHC class II as shown in FIG. 3A expression. Low-level class I staining suggested that the cells were relatively immature but did not prove that the cells were somatic stem or progenitor cells.

Using the anti-human CD34 and CD117 antibodies, which demonstrated the ability to crossreact with ovine CD34 and CD117, the ovine lung-derived cells were shown to be highly positive (>90% positive), while, in comparison, ovine peripheral blood leukocytes, as expected, were shown to have very low level staining for both of these stem cell markers as shown in FIG. 2. These data strongly suggest that the derived cells were a stem or progenitor cell population.

Western blot analysis of the SLPCs seems to support that the population is made up of immature quiescent cells that, with time and appropriate growth factor treatment, differentiated into cells that expressed CC10 and SP-C as shown in FIG. 3. These data indicate that the initial cell population had differentiated into Clara cells and type II pneumocytes from CC10- and SP-C-negative cultures, suggesting that cells initially isolated from the tissue have the ability to act as lung progenitor cells. The development of multiple types of lung cells at later stages of cell culture suggests that this population of progenitor cells is potentially multipotent or is at least a mixture of unipotent cells preprogrammed to produce many cell types. It does not appear that this initial quiescent phase has any effect on tissue growth when presented with a vascular source as shown in FIGS. 6-10. Although experiments using implanted CMFDA-labeled cell/polymer constructs seemed to suggest that all of the component parts of the lung were produced as shown in FIGS. 9 and 10, it remains unclear at this time exactly which cell or group of cells was responsible for tissue development. This is an important consideration in investigating engraftment potential of lung-derived cells into damaged and/or diseased lungs since environmental cues seem to be of critical importance in influencing cell maturation and ultimately development of complex tissues. Ongoing studies are currently underway to ascertain what cell or group of cells is responsible for the development of each component part of the lung.

In a variety of tissue engineering applications, tissue assembly by cells has been facilitated by the use of polymer scaffolds, which act as templates for cell-cell attachment and ensuing tissue development. FIG. 4 illustrates this point, as SLPCs were able to attach to PGA strands and subsequently develop into tissue which resembled normal lung morphology (as noted in the scanning electron micrographs of freeze fracture preparations of normal and engineered lung; as shown in FIG. 5). The absence of tissue growth after injection into the back of nude mice of isolated cells suspended in culture media (DMEM/F12) further confirmed the importance of using a scaffold as a vehicle to enhance tissue development. This implies that the therapeutic use of these cells requires not only differentiation into correct phenotypes but also the coordination of differentiated cells on synthetic polymer in order to facilitate the assembly of functional tissue. As such, these results demonstrate that isolated lung cell differentiation and lineage formation are necessary, but not sufficient, to ensure the development of functional tissue at the organ level.

In vivo experiments using SLPC/polymer constructs showed that PGA drives assembly of tissue differently from PF-127. Although PF-127 does not dictate any three-dimensional form for the developing tissue, it supports the cells and allows for a more natural progression of lung morphogenesis. PGA has been shown to induce an immune response in an immunocompetent host; even in immunodeficient nude mice, PGA induced a foreign body response at the site of implantation. It is generally accepted that this immune response results in hydrolysis of the fibers and loss of structural support provided by the PGA matrix, thus altering the structural integrity of the developing tissue. However in experiments that combined the use of PGA and PF-127, we feel that the foreign body response was suppressed enough to allow for tissue growth due to the presence of the polaxamer hydrogel. PF-127 has been shown to have an inhibitory effect on plasma protein absorption to microsphere surfaces with a subsequent reduction in phagocytosis and neurtrophil activation. This could potentially be why we did not see induction of a foreign body response in experiments where PGA and PF-127 were used in combination to form the SLPC/polymer constructs such as in the wedge resection experiments presented.

This study documents the existence in adult lung of a population of potentially multipotent progenitor cells that are capable of generating lung tissue when combined with a synthetic scaffold. It emphasizes the potential of scaffold-based tissue engineering approaches in combination with the use of progenitor or stem cells to generate new lung tissue. In a variety of other tissue engineering applications, tissue assembly by cells has been facilitated by the use of polymer scaffolds that act as templates for cell-cell organization. In our experiments, we found that cells not associated with scaffolds differentiated into lung-specific lineages with no evidence of tissue assembly. In contrast, cell polymer constructs generated tissue similar in morphology to normal lung (possessing the appearance of alveoli and terminal bronchi) and cells that expressed markers of Clara cells, epithelial cells, and pneumocytes. This implies that the therapeutic use of stem or progenitor cells requires not only differentiation of these cells into correct phenotypes, but also the coordination of differentiated cells into a functional assembly of tissue. Furthermore, the implantation of these quiescent cells within a specific lung milieu might encourage site-specific differentiation.

The de novo generation of lung tissue (i.e., “pneumogenesis”) raises the possibility of new therapies for treatment of lung diseases/disorders based on the delivery of progenitor cells in appropriate scaffold materials. The generation of new lung tissue from cells derived from adult lung is particularly appealing, since it offers the possibility of autologous therapy, which minimizes the risks of graft rejection and disease transmission. Future studies will focus on further characterization of progenitor cells derived from the lung, including understanding the relationship of these cells to other stem and progenitor cells as well as investigating engraftment of lung-derived adult progenitor cells into both healthy and diseased lung. The limits of the amount of harvested tissue required to enable these applications, and the suitability of diseased tissue as a source of progenitor cells for these treatments, also remain important considerations for future studies. These data also emphasize the importance of identification and characterization of somatic stem cells as well as the necessity of scaffold-based approaches for engineering tissues from stem cell sources.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A composition comprising an engineered tissue including somatic lung progenitor cells seeded onto or into a bio-compatible, bio-degradable polymer scaffold or other non biodegradable matrix adapted to restore pulmonary functions to non-functioning sites of a diseased, damaged or injured mammalian lung.

2. A composition comprising an isolated mixture of somatic lung progenitor cells capable of being differentiated into functional lung cells and grown into functional lung tissue.

3. An implantable composition comprising a bio-compatible, biodegradable polymer scaffold or other non biodegradable matrix supporting a pulmonary differentiated and functional tissue derived from a mixture of lung derived somatic progenitor cells grown on the scaffold.

4. A method comprising the steps of:
isolating a composition including a mixture of lung derived somatic progenitor cells obtained from an autologous biopsy, and
growing the composition to form a differentiated lung cell population.

5. A method comprising the steps of:
isolating a composition including a mixture of lung derived somatic progenitor cells obtained from an autologous biopsy;
depositing or seeding the isolated lung progenitor cells onto and/or into a polymer scaffold preferably a hydrogel scaffold to a form cell/polymer construct adapted to grow new and functional pulmonary tissue;
growing the cells in the cell/polymer construct to form a functional pulmonary tissue/polymer construct; and
implanting the tissue/polymer construct into non-functioning areas of an injured mammalian lung including a human lung to restore some or all of the functionality of the non-functioning area.

6. A method comprising the steps of:
isolating a composition including a mixture of lung derived somatic progenitor cells obtained from an autologous biopsy;
depositing or seeding the isolated lung progenitor cells onto and/or into a polymer scaffold preferably a hydrogel scaffold to a form cell/polymer construct adapted to grow new and functional pulmonary tissue;
implanting the cell/hydrogel construct directly into non-functioning areas of a diseased lung, damaged lung or injured lung, where cell differentiation and proliferation into functional pulmonary tissue occurs in vivo to restore some or all of the functionality of the non-functioning area. The construct can also be implanted into damaged or injured lung tissue sites to promote healing, ameliorate adverse symptoms, prevent further lung damage and to protect the sites during healing.
